(19) United States
(12) Patent Application Publication
(10) Pub. No.: US 2017/0249833 A1
(43) Pub. Date: Aug. 31, 2017

(54) Test Equipment Interface

(71) Applicant: Plato Electronics LLC, San Diego, CA (US)

(72) Inventor: Ryan Michael Johnson Shroat, San Diego, CA (US)

(21) Appl. No.: 15/442,904

(22) Filed: Feb. 27, 2017

Related U.S. Application Data
(60) Provisional application No. 62/299,603, filed on Feb. 25, 2016.

(51) Int. Cl. G08C 17/02; A61B 5/021; H04N 7/18; G06F 3/16; G06F 19/00; H04N 5/33

(52) U.S. Cl. CPC G08C 17/02; G06F 19/3406; H04N 5/33; H04N 7/185; G06F 3/167; A61B 5/021; G08C 2200/00

(57) ABSTRACT

Systems and methods for a test equipment interface are described. The test equipment interface includes a test equipment device and a wearable device that are in wireless communication with one another. The wearable device displays information collected or measured by the test equipment device. As a result, a user can operate the test equipment device while receiving measurements. The wearable device may include a smart watch, a pair of smart glasses, a cellular telephone, or a tablet. The test equipment device may include electrical test equipment, machining tool, welding machinery, or a medical measuring device. The test equipment interface may operate using voice recognition technology. Additionally, the test equipment interface may have text-to-speech functionality, such that measurements can be audibly communicated. The test equipment interface may also include an application that displays, stores, analyzes, and catalogs any information taken by the test equipment device for later review.

### CROSS-REFERENCE(S) TO RELATED APPLICATION(S)

This application claims the benefit of priority on U.S. Provisional Patent Application Ser. No. 62/299,603, filed on Feb. 25, 2016 and entitled Test Equipment Interface, the entirety of which is hereby incorporated by reference.

### BACKGROUND OF THE INVENTION

1. Field of the Invention

The present invention relates in general to the field of test equipment. More particularly, the present invent relates to a wearable device mounted to a user that is capable of displaying information taken from test equipment. Specifically, a preferred embodiment of the present invention relates to a wearable device that displays measurements taken from a multi-meter, an oscilloscope, a spectrum analyzer, a machining tool, a welding machine, a medical device and the like.

2. Discussion of the Related Art

Historically, a variety of different test equipment has been made available to measure various properties. For instance, electronic test equipment has been used to measure the electricity supplied to various components, circuits, and the like. Some examples or electronic test equipment include multi-meters, oscilloscopes, and spectrum analyzers.

Additionally, other types of tools are used to manufacture new products. For instance, a machining tool such as a cutting tool, a vertical mill, and a lathe can be used to manufacture products based on input provided by an operator. Further still, welding equipment can be used to connect different materials to one another. A user may document use of these types of equipment.

Other measurement devices may be used in a medical context. For instance, a sphygmomanometer may be used to take blood pressure readings. Other devices can be used to take other vital medical readings including a patient's pulse, temperature, or blood oxygen level. Furthermore, inclinometers or goniometers can monitor and measure angles including range of motion, passive range of motion, or other relevant information.

A common problem with all of the equipment discussed above is that the equipment requires a user to use one or both of the user's hands to operate the equipment. When the user wants to take or record a measurement, he or she must look away from the equipment. Furthermore, the user must release the equipment from one hand in order to write down measurements.

What is needed therefore is a test equipment interface that allows the user to operate various equipment while measuring and recording results from the equipment as the user continues to use the equipment. What is further needed is a wearable display that connects to the electronic test equipment wirelessly. What is also needed is a test equipment interface that allows the various equipment to be controlled using a device control interface or voice command technology.

### SUMMARY AND OBJECTS OF THE INVENTION

By way of summary, the present invention is directed to a test equipment interface including a test equipment device and a wearable device. A primary object of the invention is to provide an apparatus that allows the wearable device to be mounted to a user, where the wearable device wirelessly communicates with the test equipment device. This may be achieved using a microprocessor connected to the test equipment device to allow for wireless communication with the wearable device. The wearable device can then display information that is taken from the test equipment device and transmitted to the wearable device.

For instance, in accordance with a first aspect of the invention, the test equipment device could be electronic test equipment, for instance, a multi-meter, an oscilloscope, or a spectrum analyzer. In such an embodiment, the test equipment could take electronic readings and transmit them to the wearable device. These devices may be portable, handheld devices or non-portable bench top devices.

In accordance with a second aspect of the invention, the test equipment device could be a machining tool, for instance, a CNC machine, a cutting tool, a vertical mill, and a lathe. With these types of test equipment, the wearable device could display changes in the location of the cutting tool, for instance in a vertical, horizontal, or lateral direction.

In accordance with a third aspect of the invention, the test equipment device could be an infrared camera. The infrared camera could be attached to a helmet that is worn by the user in connection with use of a welding machine. The infrared camera could thus measure the temperature of a flame created by the welding machine, and transmit the measurement to the wearable device. Thus, the user could monitor the temperature of the flame without looking away from the welding machine and the materials being welded.

In accordance with a fourth aspect of the invention, the test equipment device could be a medical measuring device that measures vital statistics of a patient. For instance, the patient's blood pressure or pulse could be measured, and then transmitted to the wearable device. This would allow the user to maintain focus on the patient and the test equipment device while viewing and/or recording measurements. Additionally, the test equipment device could be controlled by a user, for instance using voice recognition software to enable the user to control the medical measuring device without the use of the user's hands. Alternatively, the user could use his or her hands to control the test equipment device and select the type of measurement desired.

In accordance with another object of the invention, the wearable device could be a smart watch, a pair of smart glasses, a tablet, a portable computer, or a cellular telephone, all of which are configured for wireless communication with other devices. Additionally, the wearable device may be controlled based on vocal commands from the user. The user may record measurements, control the test equipment, and adjust settings of the test equipment all based on a vocal command by the user.

In accordance with yet another object of the invention, any of the measurements displayed on the wearable device could be stored for later review.

In accordance with another object of the invention, the measurements can be audibly communicated to the user. For instance, the wearable device can audibly communicate any measurements. Alternatively, a pair of headphones that can communicate measurements may be associated with the wearable device. Alternatively still, the test equipment device could be configured to audibly communicate any measurements. Also, a pair of headphones could be connected to the test equipment device to communicate audible readings taken by the test equipment. Other speakers could be used with either the wearable device or the test equipment to audibly communicate readings and measurements to a user. As a result, the user can stay focused on what is being measured.

In accordance with another object of the invention, the test equipment device could be a wireless tool. Different criteria can be measured and stored relating to use of the wireless tool. For instance, the, test equipment device could be a wireless drill. While in use, criteria such as amount of use, time of use, and amount of load can be measured and stored. This would allow a user to anticipate when a given drill bit would likely become dull. Additionally, this would allow business owners such as contractors to monitor productivity of workers based on the amount of time a given tool is in use. Additionally, profiles can be set that establish maximum criteria values. For instance, the hand drill could automatically be powered off in the event that it experiences a significant load.

These and other aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

A clear conception of the advantages and features constituting the present invention, and of the construction and operation of typical mechanisms provided with the present invention, will become more readily apparent by referring to the exemplary, and therefore non-limiting, embodiments illustrated in the drawings accompanying and forming a part of this specification, wherein like reference numerals designate the same elements in the several views, and in which:

FIG. 1 illustrates a test equipment interface including a smart watch and a handheld multi-meter according to the present invention;

FIG. 2 illustrates a test equipment interface including a pair of smart glasses and a bench top multi-meter according to the present invention;

FIG. 3 illustrates a test equipment interface including a cellular telephone and an oscilloscope according to the present invention;

FIG. 4 illustrates a test equipment interface including a tablet and a spectrum analyzer according to the present invention;

FIG. 5 illustrates a test equipment interface including a pair of smart glasses and a lathe according to the present invention;

FIG. 6 illustrates a test equipment interface including a pair of smart glasses and a vertical mill according to the present invention;

FIG. 7 illustrates a test equipment interface including a pair of smart glasses and a computerized numerical control machine according to the present invention;

FIG. 8 illustrates a test equipment interface including a welding helmet with an infrared camera according to the present invention; and

FIG. 9 illustrates a test equipment interface including a medical measuring device according to the present invention.

In describing the preferred embodiment of the invention which is illustrated in the drawings, specific terminology will be resorted to for the sake of clarity. However, it is not intended that the invention be limited to the specific terms so selected and it is to be understood that each specific term includes all technical equivalents which operate in a similar manner to accomplish a similar purpose. For example, the word connected, attached, or terms similar thereto are often used. They are not limited to direct connection but include connection through other elements where such connection is recognized as being equivalent by those skilled in the art.

### DESCRIPTION OF PREFERRED EMBODIMENTS

The present invention and the various features and advantageous details thereof are explained more fully with reference to the non-limiting embodiments described in detail in the following description.

### 1. SYSTEM OVERVIEW

As discussed above, the prior art has failed to offer various test equipment that can be used with a wearable device capable of displaying measurements or readings relating to the test equipment. The proposed invention connects a wearable display device to electronic test equipment wirelessly. For instance, readings from a handheld multi-meter could be displayed on a smart watch. Similarly, a bench top multi-meter could be connected to a head mounted display, such as a pair of smart glasses like Google Glass. Further still, a machining tool, such as a vertical mill or lathe, could be connected to a wearable display to allow a user to focus on a piece being machined, while seeing changes in tool position. Other test equipment could be used with wearable devices to allow a user to view measurements from the test equipment while continuing to operate the test equipment. Additionally, the test equipment and/or wearable device may feature a text-to-speech function. This allows a user to hear the measurement results while allowing the user to maintain focus on what is being measured.

### 2. Detailed Description of Preferred Embodiments

Referring initially to the Figures, a test equipment interface 20 is illustrated. The test equipment interface 20 may include a test equipment device 22 and a wearable device 24 that can be mounted to a user 26. The test equipment device 22 is in wireless communication with the wearable device 24 such that information including measurements, readings, and the like can be transmitted to the wearable device 24. The measurements, readings, and the like could also be communicated audibly to the user through speakers, headphones, or other headsets associated with either the test equipment device 22 or the wearable device 24. The wearable device 24 can then display, save, and organize the information that is transmitted to it wirelessly from the test equipment 20. Additionally, the wearable device 24 can be used to control or select the data to be measured, or otherwise control the test equipment device 22. This allows the user 26 to focus on a task while still receiving the information provided by the test equipment device 22. The test equipment interface 20 also allows a user 26 to have an increased freedom of movement, while maintaining records of any measurements that are taken. The wearable device 24 may also be equipped with voice recognition software to allow the user 26 to optimize use of the test equipment device 22 as will further be described below.

Specific embodiments of the present invention will now be further described by the following, non-limiting examples, which will serve to illustrate various features of significance. The examples are intended merely to facilitate an understanding of ways in which the present invention may be practiced and to further enable those of skill in the art to practice the present invention. Accordingly, the examples should not be construed as limiting the scope of the present invention. For instance, the test equipment device 22 may be a handheld multi-meter 30, a bench top multi-meter 44, an oscilloscope 50, a spectrum analyzer 52, a machining tool 54, a computerized numerical control (CNC) machine 56, a vertical mill 58, a lathe 60, an infrared camera 62, a medical measuring device 72, or any other device that measures or documents information. Additionally, examples of potential wearable devices 24 include, smart watches 32, smart glasses 34, a head mounted display 33, tablets 36, portable computers, cellular telephones 40, and any other device capable of displaying information. Exemplary application of some of these test equipment devices 22 and wearable devices 24 will be provided below although it is understood that any of the test equipment devices 22 could be used with any wearable devices 24.

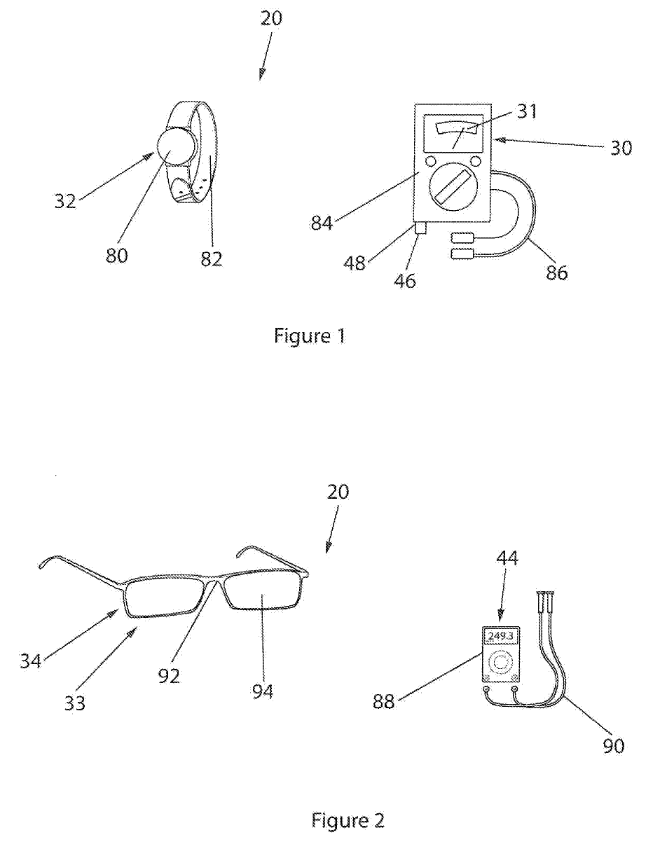

Looking initially to FIG. 1, a first potential embodiment of the test equipment interface 20 is shown with a smart watch 32 as the wearable device 24 being used with a handheld multi-meter 30 as the test equipment device 22. As known to one of ordinary skill in the art, a smart watch 32 includes a computer with hardware and software configured to communicate wirelessly with other devices as well as the internet. Also known to one of ordinary skill in the art, a smart watch 32 may comprise a Bluetooth or Wi-Fi connected wearable device that is capable of running application programs. For example, the, smart watch 32 could be an Android Wear or Apple Watch. Like common watches, the smart watch 32 may include a display 80 and a band 82 that allows the smart watch 32 to be secured to the user 26. Handheld multi-meters 30 are generally understood by those of ordinary skill in the art to include a body 84 that displays an electronic reading 31 and test leads 86 that are used to help diagnose electrical problems with a wide variety of devices, including electronic equipment, motor controls, appliances, power supplies, and other devices containing wiring. Other handheld devices could be used that measure voltage, current (alternating or direct), resistance, capacitance, continuity, inductance, temperature, or frequency. When the handheld multi-meter 30 is in use, the test leads 86 are used to engage two ends of a circuit or component. The handheld multi meter 30 can then measure the voltage, current, and resistance using equipment located within the body 84 and display the measurement of the electronic reading 31.

While the illustrated embodiment shows a handheld multi-meter 30 with an analog display, it should be understood that other handheld multi-meters 30 could be used including ones with a digital display. However, considering the measurements provided by the handheld multi-meter 30 will be displayed on the wearable device 24, the handheld multi-meter 30 need not have a display if desired.

In use, the test equipment interface 20 allows a user 26 to hold the test leads 86 of the handheld multi-meter 30 against an object or circuit to be tested, and read the electronic reading 31 measurements taken from the handheld multi-meter 30 that are transmitted to the wearable device 24 without breaking eye contact with the object or circuit being tested. For instance, the wrist of the user 26 may be located directly adjacent to the test leads 86 such that the user 26 can read the information displayed on the smart watch 32. Alternatively, when the wearable device 24 is a pair of smart glasses 34, the information will be displayed directly in the line of sight of the user 26. As known to one of ordinary skill in the art, smart glasses 24 are wearable devices that connect wirelessly to the internet and other devices and can run an application such as an Android Wear application or other applications to display a measurement result. As also known to one of ordinary skill in the art, the smart glasses 34 include a frame 92 that engages the head of a user 26, and a transparent glass 94 that displays information, but allows the user 26 to see through the transparent glass 94. As a result, the handheld multi-meter 30 does not need to be readily viewable when the multi-meter 30 is in use, but instead could be located remotely for instance on a user's belt (not shown). The user 26 could then more easily and conveniently take readings while moving around a test area. The user 26 can walk around freely while taking measurements and recording results as he or she goes.

Alternatively, a bench top multi-meter 44 can be used as the test equipment device 22. Generally speaking, while lacking the convenience and mobility that a handheld multi-meter 30 can offer, a bench top multi-meter 44 can provide similar measurements with a very high degree of accuracy. Additionally, the bench top multi-meter 44 allows a user 26 to take measurements on very small test points that require focus and concentration. Similarly, bench top multi-meters 44 usually show more precise results with more significant digits than a handheld multi-meter. The bench top multi-meter 44 also features a body 88 with test leads 90 that can be connected to an object or a circuit to measure the voltage, current (alternating or direct), resistance, capacitance, continuity, inductance, temperature, or frequency. The bench top multi-meter 44 could be used with any of the wearable devices 24, for instance a pair of smart glasses 34 as shown in FIG. 2. Smart glasses 34 are especially desired where measurements are being taken about small test points so that the user 26 would not have to break eye contact to view the measurement.

Again, while the illustrated embodiment shows a bench top multi-meter 44 with a digital display, it should be understood that other bench top multi-meters 44 could be used including ones with a analog display. However, considering the measurements provided by the bench top multi-meter 44 will be displayed on the wearable device 24, the bench top multi-meter 44 need not have a display if desired.

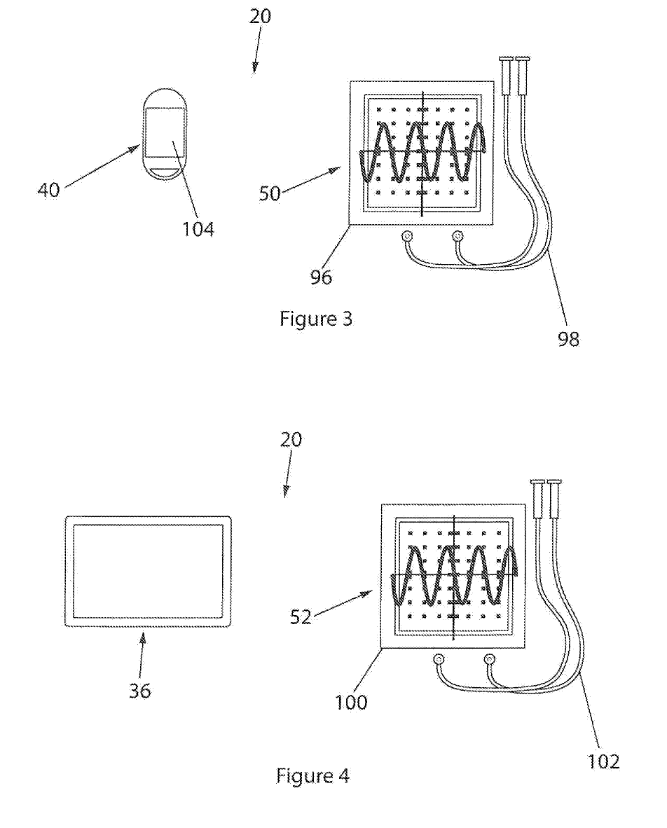

Other electronic devices could serve a the test equipment device 22. For instance, an oscilloscope 50 could be used to monitor changes in electrical signal as a function of time of a circuit or an object as shown in FIG. 3. As known to one of ordinary skill in the art, an oscilloscope 50 is a device that shows time domain signals and measures amplitude, frequency, and wave shape of signals. The resulting information is then transmitted to the wearable device 24, as shown on a display 104 of a cellular phone 40. Like the handheld multi-meter 30 and the bench top multi-meter 44, the oscilloscope 50 features a body 96 and test leads 98. Further still, a spectrum analyzer 52 may be used as the test equipment device 22 to measure the magnitude of an input signal versus frequency within the full frequency range of a circuit or an object. Spectrum analyzers 52 are capable of displaying frequency domain measurements and can show frequency, amplitude, and bandwidth of such signals. As shown in FIG. 4, the spectrum analyzer 52 features a body 100 and test leads 102 and is used with a tablet 36 as the wearable device 24.

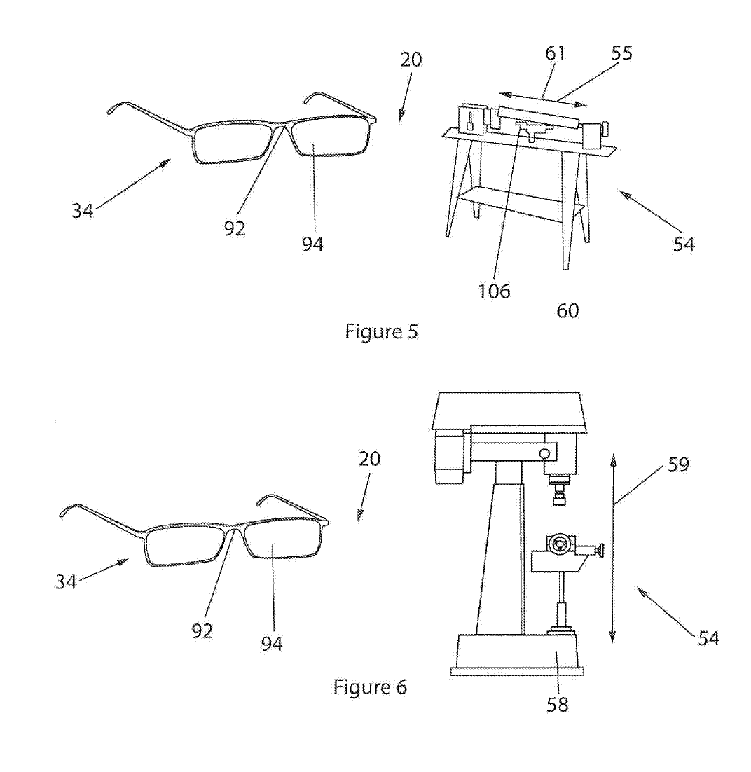

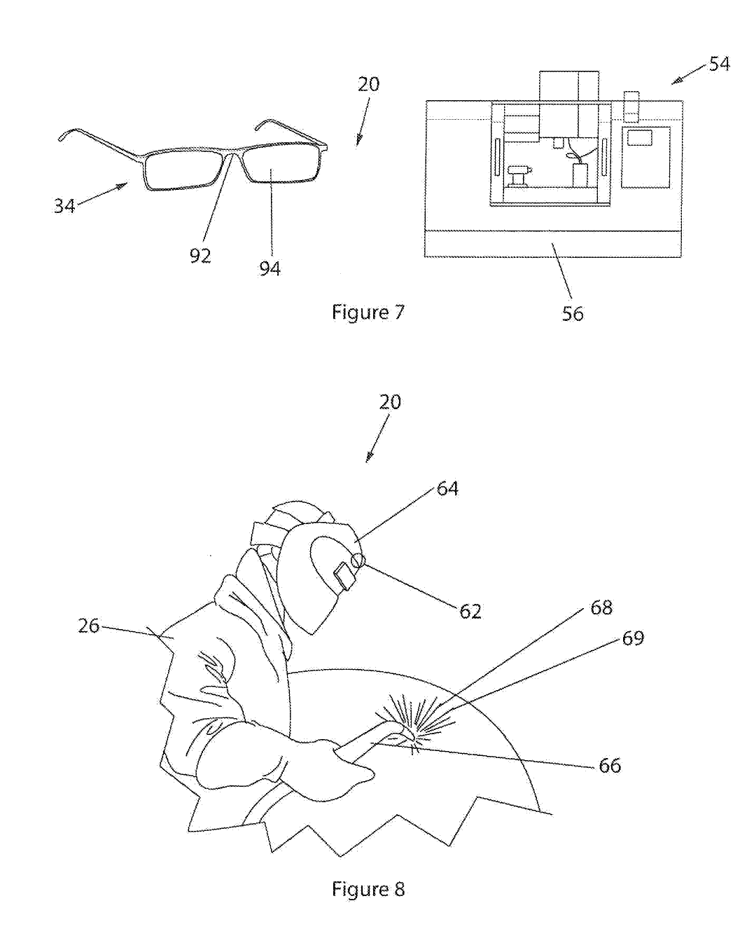

Alternatively still, the test equipment devices 22 could be a cutting tool or machining tool 54, such as a vertical mill 58, a lathe 60 or another cutting tool such as a computerized numerical control (CNC) machine 56. Looking initially to FIG. 5, a lathe 60 is shown being used with smart glasses 34. As known to one of ordinary skill in the art, a lathe 60 rotates a work piece 106 about a central axis and trims the work piece 106 into a desired configuration. In using the test equipment interface 20 with a lathe 60, the user 26 can monitor the horizontal position 55 of the lathe 60 and the changes made to the work piece 106. For instance, the smart glasses 34 can monitor the change of position 61 or movement of a number of different components, including the speed of the machining tool 54, as well as the change of position of a tool (not shown) about the machining tool 54. More specifically, the smart glasses 34 monitor the position of the tool about a fixed origin. Alternatively still, an origin can be set so that the amount of material removed from a work piece 106 can be measured. Similarly, a vertical mill 58 is used with smart glasses 34 as shown in FIG. 6 to measure changes in vertical position 59. Further still, a CNC machine 56 could be used with smart glasses 34 as shown in FIG. 7. The smart glasses 34 could display the change in tool position, rotations per minute of the machining tool 54, or amount of material removed. Any of these measurements could be helpful to allow a user to monitor changes about the work piece 106 and the machining tool 54. For these types of test equipment devices 22, smart glasses 34 are preferred such that the user 26 can monitor changes while keeping his or her line of sight focused on the machining tool 54 and the work piece 106 being cut.

In yet another embodiment, the test equipment device 22 could be an infrared camera 62. As shown in FIG. 8, the infrared camera 62 could be used in conjunction with a welding helmet 64. More specifically, the infrared camera 62 measures the temperature 69 of a flame 68 created by a welding machine 66. The temperature of the flame 68 could then be transmitted to the wearable device 24. Preferably, the wearable device 24 is a pair of smart glasses that the user 26 is wearing underneath the welding helmet 64. Alternatively, the wearable device 24 may be a display located on the inside of the helmet, for instance, a small LCD screen (not shown) that is within the operator's field of vision and mounted to the welding helmet 64 to display the temperature of the flame. As a result, the user 26 can monitor the temperature of the flame 68 and adjust the welding machine 66 as needed to improve the quality of the weld. As known to one of ordinary skill in the art, two pieces of metal must reach a minimum temperature before being successfully welded together. Furthermore, a user may identify a specific temperature, through experience or trial-and-error, that results in a higher quality weld.

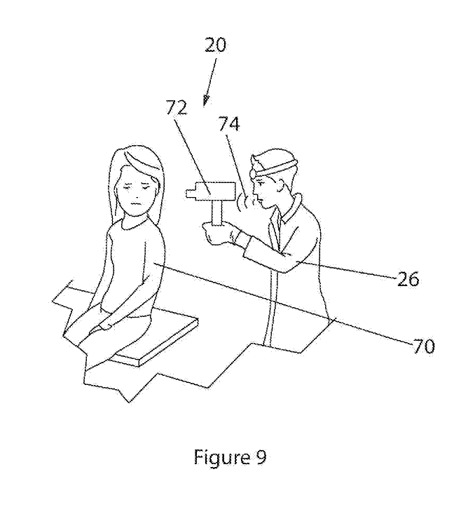

In another embodiment, the test equipment, device 22 could be medical measuring devices 72 as seen in FIG. 9, for instance a sphygmomanometer (device that takes blood pressure readings), or a device that takes other vital medical readings including a patient's 70 pulse, temperature, or blood oxygen level. Also, the medical measuring device 72 may be used to display angles in the user's field of vision so that the user can monitor range of motion, passive range of motion, goniometry, or other relevant information, including for instance inclinometers or goniometers. Again, by having test equipment devices 22 that communicate information to a wearable device 24, the user 26 can more easily process and record measurements without having to look away from the patient 70.

Alternatively still, the test equipment device 22 could be a wireless tool that is wireless communication with a wearable device 24. This would allow various criteria associated with the wireless tool to be measured and stored for future review and analysis. For instance, the wireless tool could be a wireless drill. When in use, different features of the drill could be measured and stored, such as time of use and amount of load. This would allow a user to anticipate when a given drill bit will likely become dull, such that he or she can take proactive measures to replace the hit. Similarly, a profile can be set for the wireless tool such that the tool will automatically be disabled if certain maximum criteria are met. This protects the tool from damage while also increasing safety of the user. Additionally, by tracking the amount of use of the wireless tool, business owners such as contractors can better appreciate productivity of workers. Where the tool is not used for extended periods of time, the contractor can then investigate whether the employee was performing another task or if they were wasting time.

The test equipment devices 22 may be equipped with a microprocessor 46 or other device that enables the test equipment device 22 to communicate with the wearable device 24. In some instances, the microprocessor 46 connects through a port, for instance, a GPIB port. Alternatively, the test equipment device 22 could also be equipped with a USB port capable of accepting a device that enables wireless signals be transmitted and received. The microprocessor 46 may than transmit information wirelessly, for instance by Bluetooth, Wi-Fi, or cellular connection that enables remote access from anywhere, from the test equipment device 22 to the wearable device 24. As a result, any information or measurements can be stored or accessed remotely, for instance in an internet cloud. The microprocessor 46 can perform calculations on measurements. For instance, where the test equipment device 22 is a handheld multi-meter 30, the microprocessor 46 may indicate that a measurement is within a certain range. Similarly, where the test equipment device 22 is an oscilloscope, or spectrum analyzer, the microprocessor 46 may confirm that a signal conforms to a certain waveshape or is within a certain range.

Additionally, the test equipment devices 22 need not communicate directly with the wearable device 24. For instance, the test equipment device 22 may send information first to another device, and the other device then transmits the information to the wearable device 24. In one embodiment, the test equipment device 22 may first transmit information to a cellular telephone 40, which then communicates the information to a smart watch 32 or a pair of smart glasses 34. Again, the information can also be communicated audibly from the test equipment device 22 or the wearable device 24 via speakers, headsets, or headphones.

The test equipment interface 20 may include an application that is downloadable to the wearable device 24. The application may include software that allows any information processed by the test equipment interface 20 to be displayed, stored, analyzed, cataloged, or the like. The application may allow the test equipment interface 20 to perform calculations on measurements. For instance, where the test equipment device 22 is a handheld multi-meter 30, application may indicate that a measurement is within a certain range. Similarly, where the test equipment device 22 is an oscilloscope, or spectrum analyzer, the application may confirm that a signal conforms to a certain waveshape or is within a certain range. The application may allow the user 26 to review measurements and compare against previous measurements.

Of course, a single test equipment device 22 may be used with a single wearable device 24. Alternatively, multiple test equipment devices 22 could be used with a single wearable device 24. Further still, a single test equipment device 22 could be used with multiple wearable devices 24. Any variety of configurations would be possible based on the desire of the user.

The wearable device 24 may also be equipped with voice recognition software such that the user 26 can provide a vocal command 74 for the wearable device 24 to display, save, and organize the information from the test equipment device 22. The voice recognition software further allows a user 26 to adjust the test equipment device 22 settings audibly while remaining focused on the measurement or activity. For instance, with an oscilloscope 50, the user 26 can audibly adjust the volts/division settings or time/division settings as desired. Similarly, the user 26 can adjust other settings, including AC or DC coupling, vertical level adjustments for DC coupling, and trigger configuration (rising edge, falling edge, or delay). Further still, voice recognition software can be used to switch between single shot or free running display, perform math functions on signal, and turn on or off measurements such as frequency or duty cycle. Use of voice recognition software is especially helpful where the user 26 is using both of his or her hands to control the test equipment device 22.

Although the best mode contemplated by the inventors of carrying out the present invention is disclosed above, practice of the present invention, is not limited thereto. It will be manifest that various additions, modifications and rearrangements of the features of the present invention may be made without deviating from the spirit and scope of the underlying inventive concept.

For example, any one of the above-described lest equipment devices 22 may be used with any one of the wearable devices 24. Additional test equipment devices 22 and wearable devices 24 could also be used in order to improve the convenience, effectiveness, and safety of taking measurements of different test equipment devices 22.

Moreover, the illustrations provide exemplary, test equipment interfaces 10, and the individual components need not be formed in the disclosed shapes, or assembled in the disclosed configuration, but could be provided in virtually any shape, and assembled in virtually any configuration. Further, although the test equipment devices 22 and wearable devices 24 described herein are physically separate, the test equipment devices 22 and wearable devices 24 could also be physically connected. Additionally, while the above description specifically discusses Bluetooth wireless connection between the components, it should be understood that any wireless communication could be employed to connect the test equipment devices 22 and the wearable devices 24. Furthermore, all the disclosed features of each disclosed embodiment can be combined with, or substituted for, the disclosed features of every other disclosed embodiment except where such features are mutually exclusive.

It is intended that the appended claims cover all such additions, modifications and rearrangements. Expedient embodiments of the present invention are differentiated by the appended claims.

What is claimed is:

1. A test equipment interface comprising:
a test equipment device;
a wearable device mounted to a user in wireless communication with the test equipment device, the wearable device capable of displaying information taken from the test equipment device that is transmitted wirelessly from the test equipment device to the wearable device.

2. The test equipment interface of claim 1, wherein the test equipment device is a handheld multi-meter configured to take electronic readings.

3. The test equipment interface of claim 2, wherein the wearable device is selected front a group consisting of a smart watch, a pair of smart glasses, a tablet, a cellular phone, and a head-mounted display.

4. The test equipment interface of claim 3, wherein the wearable device displays an electronic reading from the handheld multi-meter.

5. The test equipment interface of claim 1, wherein the test equipment device is a bench top multi-meter.

6. The test equipment interface of claim 5, further comprising a microprocessor connected to the bench top multi-meter through a GPIB port.

7. The test equipment interface of claim 6, wherein the microprocessor is enabled for Bluetooth connection with the wearable device.

8. The test equipment interface of claim 1, wherein the test equipment device is selected from a group consisting of an oscilloscope, a spectrum analyzer, a machining tool, an infrared camera, and a medical measuring device that measures a vital statistic of a patient.

9. The test equipment interface of claim 8, wherein the machining tool is selected from a group consisting of a cutting tool, a vertical mill, and a lathe.

10. The test equipment interface of claim 9, wherein the wearable device displays a change in position of the machining tool.

11. The test equipment interface of claim 8, further comprising:
a helmet used by the user with the infrared camera attached to the helmet; and
a welding machine.

12. The test equipment interface of claim 11, wherein the infrared camera can measure a temperature of a flame created by the welding machine.

13. The test equipment interface of claim 8, wherein the test equipment device is connected to the wearable device using Bluetooth enabled microprocessor.

14. The test equipment interface of claim 8, wherein the medical measuring device measures the patient's blood pressure.

15. The test equipment interface of claim 8, wherein the medical measuring device measures the patient's pulse.

16. The test equipment interface of claim 1, wherein the wearable device can be controlled using a vocal command.

17. The test equipment interface of claim 16, wherein the wearable device can record measurements based on the vocal command by the user.

18. The test equipment interface of claim 16, wherein the wearable device can control the test equipment based on the vocal command by the user.

19. The test equipment interface of claim 16, wherein the wearable device can adjust settings of the test equipment based on the vocal command by the user.

20. The test equipment interface of claim 1, further comprising means for audibly communicating measurements collected by the test equipment device.
